(19) United States
(12) Patent Application Publication
(10) Pub. No.: US 2023/0279275 A1
(43) Pub. Date: Sep. 7, 2023

(54) HOT MELT ADHESIVE COMPOSITION

(71) Applicant: BOSTIK SA, Colombes (FR)

(72) Inventors: Genta OKAZAKI, Osaka (JP); Xin YE, Minhang District Shanghai (CN)

(21) Appl. No.: 18/005,679

(22) PCT Filed: Jul. 19, 2021
(86) PCT No.: PCT/EP2021/070078

§ 371 Date: Jan. 17, 2023

(30) Foreign Application Priority Data
Jul. 21, 2020 (EP) 20186910.4

(51) Int. Cl. C09J 153/02

(52) U.S. Cl. CPC C09J 153/02

(57) ABSTRACT

The present invention relates to a hot melt adhesive composition comprising styrene triblock copolymer components, and related articles comprising at least one interior or exterior surface coated with the hot melt adhesive composition.

### TECHNICAL FIELD

The present invention relates to a hot melt adhesive composition comprising styrene triblock copolymer components, and related articles comprising at least one interior or exterior surface coated with the hot melt adhesive composition.

### TECHNICAL BACKGROUND

Hot melt adhesives (HM adhesives) are substances which are solid at room temperature and which often comprise neither water nor solvent. They are applied in the molten state after heating to a temperature generally of between 100 and 250° C., most often between 130 and 180° C., and solidify during cooling, thus forming a seal (or adhesive joint) which ensures the attachment of the two substrates to be assembled. Hot melt adhesives are generally provided in the form of compositions which comprise a thermoplastic polymer and optionally a tackifying resin and a plasticizer.

Hot melt adhesives are widely used in various applications, such as packaging, bookbinding, and woodworking, as they have good stability and do not require the use of solvents. Such adhesives can also be used in nonwoven applications. For example, disposable nonwoven absorbent articles are widely used for infant, young child, incontinent adult, and feminine care applications.

Typical disposable nonwoven absorbent articles include disposable diapers, training pants, adult incontinent pads and briefs, feminine sanitary napkins or pads, surgical masks. These items aim at receiving and containing body fluids and are usually worn against or in close proximity to the skin.

To manufacture disposable nonwoven absorbent articles, hot melt adhesive compositions are generally used to bond together various substrates of said articles, as they provide rapid bonding compared to other adhesives such as water-based or solvent-based adhesive compositions, which require a drying step of the water or solvent.

Hot melt adhesive compositions suitable for this purpose should possess the appropriate bond strength to adhere the substrates involved. More particularly such hot melt adhesives should present a good adhesion in a variety of conditions. Document WO 2012/006132 A2 relates to a hot-melt adhesive for absorbent articles comprising a thermally reversible block copolymer that comprises a polymer of vinyl aromatic hydrocarbon and a conjugate diene compound, and a α-methylstyrene resin.

Document WO 2019/069610 A1 relates to an adhesive composition comprising a thermoplastic polymer as a base polymer, the composition being devoid of an α-methyl styrene-based resin.

Document JP 2008/297441 A relates to a hot melt adhesive comprising a hydrogenated thermoplastic block copolymer, which is a copolymer of a vinyl aromatic hydrocarbon and a conjugated diene compound, a tackifying resin, and a plasticizer, wherein the 10 wt % toluene viscosity of the hydrogenated thermoplastic block copolymer at 30° C. is 100 to 2,600 mPa·s.

Document JP 2016/065121 A relates to hot melt adhesive comprising a thermoplastic block copolymer which is a copolymer of a vinyl-based aromatic hydrocarbon and a conjugated diene compound, having a styrene content from to 45% by weight and a diblock content from 50 to 90% by weight, and a radial type styrene block copolymer having a viscosity of a 25 wt % toluene solution at 25° C. of 250 mPa·s or less.

There is thus a need for a hot melt adhesive composition which provides good adhesion, particularly in nonwoven material-based products in order to receive and contain bodily fluids. More particularly, there is a need for a hot melt adhesive composition which provides good adhesion at a variety of temperatures and in a variety of different coating weights. Thus, there is a need for a hot melt adhesive composition which presents a stable peel strength depending on temperature and add-on fluctuations, particularly in nonwoven material-based products.

### SUMMARY OF THE INVENTION

It is a first object of the invention to provide a hot melt adhesive composition comprising:
a first polymer component consisting of at least one hydrogenated linear styrene triblock copolymer;
a second polymer component consisting of at least one hydrogenated linear styrene triblock copolymer having a diblock content from 10 to 90%, preferably from 20 to 80%;
at least one tackifying resin; and
at least one plasticizer;
wherein the composition comprises a weight ratio between the first polymer component and the second polymer component of 1:1 or more, preferably from 3:2 to 10:1, more preferably from 2:1 to 8:1;
wherein the composition comprises a weight ratio between the plasticizer and the total polymer components of 2.6:1 or more.

In some embodiments, the hydrogenated linear styrene triblock copolymer of the first polymer component is chosen from the group consisting of SEBS, SEPS, SEEPS, SBBS and blends thereof; preferably from the group consisting of SEBS, SBBS and blends thereof; more preferably the copolymer is SEBS.

In some embodiments, the hydrogenated linear styrene triblock copolymer of the second polymer component is chosen from the group consisting of SEBS, SEPS, SEEPS, SBBS, and blends thereof; preferably from the group consisting of SEBS, SBBS and blends thereof; more preferably the copolymer is SEBS.

In some embodiments, the polymer components have a viscosity of a 10% by weight toluene solution at 30° C. of 100 mPa·s or less.

In some embodiments, the first polymer component has a styrene content from 10 to 50%, preferably from 20 to 40%.

In some embodiments, the second polymer component has a styrene content from 5 to 50%; preferably from 10 to 40%.

In some embodiments, it comprises at least one additional polymer component comprising an additional styrene block copolymer chosen from the group consisting on SB, SI, SBS, SIS, SIBS.

In some embodiments, the composition comprises a coefficient in variation of the peel strength depending on the temperature being lower than 0.4.

In some embodiments, the composition comprises a coefficient in variation of the peel strength depending on the add-on being lower than 0.4.

In some embodiments, the composition is obtained by mixing the first polymer component, the second polymer component, the tackifying resin and the plasticizer.

It is a second object of the present invention to provide the use of the composition as described above, as an adhesive for bonding two substrates together, or as a coating on the surface of a substrate.

It is a third object of the present invention to provide and article comprising at least one interior or exterior surface coated with the hot melt adhesive composition as described above.

In some embodiments, the interior or exterior surface is a nonwoven fabric.

In some embodiments, the article is chosen from a disposable diaper, disposable training pants, a feminine sanitary napkin, an adsorbent pad, a surgical mask, and a surgical coat.

The present invention makes it possible to address the need mentioned above. In particular, the invention provides a hot melt adhesive composition which provides good adhesion at a variety of temperatures and in a variety of different coating weights, particularly in nonwoven material-based products in order to receive and contain bodily fluids. This is made possible by the hot melt adhesive composition of the present invention. In particular, it has been surprisingly found that a hot melt adhesive composition according to the present invention makes it possible to provide articles that present a stable peel strength depending on temperature and/or add-on fluctuations, in particular in nonwoven material-based products.

### DESCRIPTION OF EMBODIMENTS

The invention will now be described in more detail without limitation in the following description.

### Definitions

By “hot melt” is meant that the adhesive composition requires to be heated at least at 120° C., preferably at least at 140° C., to be applied on a substrate. The hot melt adhesive composition is thus solid at 23° C.

By “substantially free of” is meant less than 1%, preferably less than 0.1%, more preferably less than 0.01%, still more preferably about 0%, by weight of a compound or the hot melt adhesive composition.

By “styrene triblock copolymer” is meant a copolymer comprising two terminal styrene blocks and a mid-chain block formed from at least one type of monomers other than styrene monomers.

By “styrene diblock copolymer” is meant a copolymer comprising one terminal styrene block and one block formed from at least one type of monomers other than styrene monomers.

By “diblock content” is meant the weight proportion of diblock copolymers in a polymer component.

By “styrene content” is meant the weight proportion of styrene units in a block copolymer.

By “SBBS” is meant a styrene-butadiene-butylene-styrene triblock copolymer.

By “SBS” is meant a styrene-butadiene-styrene triblock copolymer.

By “SEBS” is meant a styrene-ethylene-butylene-styrene triblock copolymer.

By “SEPS” is meant a styrene-ethylene-propylene-styrene triblock copolymer.

By “SEEPS” is meant a styrene-ethylene-ethylene-propylene-styrene triblock copolymer.

By “SIBS” is meant a styrene-isoprene-butadiene-styrene triblock copolymer.

By “SIS” is meant a styrene-isoprene-styrene triblock copolymer.

By “SBC” is meant a copolymer comprising at least one styrene block, including styrene triblock copolymers and styrene diblock copolymers.

By “SB” is meant a styrene-butadiene diblock copolymer.

By “SI” is meant a styrene-isoprene diblock copolymer.

By “SEB” is meant a styrene-ethylene-butylene diblock copolymer.

By “SEP” is meant a styrene-ethylene-propylene diblock copolymer.

By “viscosity of a 10% by weight toluene solution at 30° C.” is meant the viscosity of a solution having a concentration of 10% by weight using toluene as a solvent at 30° C. The viscosity is measured by using a Brookfield viscometer.

Hot Melt Adhesive Composition

In a first aspect, the present invention relates to a hot melt adhesive composition comprising a first polymer component, a second polymer component, a tackifying resin and a plasticizer. It may also comprise other additional components.

First Polymer Component

The hot melt adhesive composition comprises a first polymer component.

The first polymer component consists of a hydrogenated linear styrene triblock copolymer. The first polymer component is substantially free of diblock content.

The hydrogenated linear styrene triblock copolymer of the first polymer component may be chosen from the group consisting of SEBS, SEPS, SEEPS, SBBS and blends thereof; preferably from the group consisting of SEBS, SBBS and blends thereof; more preferably the copolymer is SEBS.

In a preferred embodiment, the first polymer component only consists in one type of hydrogenated linear styrene triblock copolymer. In an alternative embodiment, the first polymer component is a blend of at least two hydrogenated linear styrene triblock copolymers.

The first polymer component may have a styrene weight content from 10 to 50%, preferably from 20 to 40%, by total weight of the first polymer component.

The first polymer component may have a viscosity of 10% by weight toluene solution at 30° C. (10 wt % TSV@30C) of 100 mPa·s or less, preferably from to 90 mPa·s. The viscosity is measured by using a Brookfield viscometer.

For example, the first polymer component is available under the tradenames Kraton® G-1650 and Kraton® G-1652 supplied by Kraton™ or Taipol® SEBS 6153 supplied by TSRC Corporation/Dexco Polymers.

The hot melt adhesive composition may comprise from 5 to 20%, preferably from 6 to 17%, more preferably from 7 to 14%, by weight of the first polymer component relative to the total weight of the composition.

Second Polymer Component

The hot melt adhesive composition comprises a second polymer component.

The second polymer component consists of a hydrogenated linear styrene triblock copolymer having a diblock content from 10 to 90%, preferably from 20 to 80%. In one embodiment, the hydrogenated linear styrene triblock copolymer of the second polymer component has a diblock content from 10 to 49%, preferably from 20 to 40%. In an alternative embodiment, the hydrogenated linear styrene triblock copolymer of the second polymer component has a diblock content from 50 to 90%, preferably from 60 to 80%.

The hydrogenated linear styrene triblock copolymer of the second polymer component may be chosen from the group consisting of SEBS, SEPS, SEEPS, SBBS, and blends thereof; preferably from the group consisting of SEBS, SBBS and blends thereof; more preferably the copolymer is SEBS.

In a preferred embodiment, the second polymer component only consists in one type of hydrogenated linear styrene triblock copolymer. In an alternative embodiment, the second polymer component is a blend of at least two hydrogenated linear styrene triblock copolymers.

The second polymer component may have a styrene weight content from to 50% and preferably from 10 to 40%, by total weight of the second polymer component.

The second polymer component may have a viscosity of a 10% by weight toluene solution at 30° C. (10 wt % ATSV@30C) of 100 mPa·s or less, preferably from to 100 mPa·s. The viscosity is measured by using a Brookfield viscometer.

For example, the second polymer component may be available under the tradename Kraton® G-1657, Kraton® G-1726 and Kraton® G-1924 supplied by Kraton™, or Globalprene® 9557 and Globalprene® 9526 supplied by LCY.

The hot melt adhesive composition may comprise from 1 to 10%, preferably from 1 to 8%, more preferably from 2 to 5%, by weight of the second polymer component relative to the total weight of the composition.

Weight Ratio Between the First Polymer Component and the Second Polymer Content

The hot melt adhesive composition may comprise a weight ratio between the first polymer component and the second polymer component of 1:1 or more, preferably from 3:2 to 10:1, more preferably from 2:1 to 8:1.

Additional Polymer Components

The hot melt adhesive composition may comprise an additional polymer component. In an alternative embodiment, the hot melt adhesive composition may be substantially free of any additional polymer component.

The additional polymer component may comprise an additional styrene block copolymer (SBC), other than the above-mentioned hydrogenated linear styrene triblock copolymers of the first and the second polymer components. Such additional polymer components are available in a number of different chemical and structure types. Examples of suitable additional styrene block copolymers are chosen from the group consisting on SB, SI, SBS, SIS, SIBS; preferably SIS.

If present, the hot melt adhesive composition may comprise from 0.1 to 10%, preferably from 1 to 5%, by weight of an additional polymer component relative to the total weight of the composition.

Total Polymer Components

The hot melt adhesive composition may comprise from 6% to 30%, more preferably from 7% to 25%, more preferably from 9% to 19%, by weight of total polymer components relative to the total weight of the composition.

Tackifying Resin

The hot melt adhesive composition comprises a tackifying resin (tackifier). The hot melt adhesive composition may comprise from 35 to 75%, preferably from 45 to 70%, more preferably from 50 to 60%, by weight of a tackifying resin relative to the total weight of the composition.

Suitable tackifying resins are those which are compatible with the polymer components, which extend adhesive properties, and which improve specific adhesion. As used herein, the term “tackifying resin” include:
Aliphatic and cycloaliphatic petroleum hydrocarbon resins having Ring and Ball softening points from 90° C. to 160° C. as determined by ASTM method E28-58T, the latter resins resulting from the polymerization of monomers consisting primarily of aliphatic and/or cycloaliphatic olefins and diolefins; also included are the hydrogenated aliphatic and cycloaliphatic petroleum hydrocarbon resins, examples of such commercially available resins based on a C5 olefin fraction of this type are Piccotac 95 tackifying resin sold by Eastman chemical and Escorez 1310LC sold by ExxonMobil Chemical Company;
Aromatic petroleum hydrocarbon resins and the hydrogenated derivatives thereof;
Aliphatic/aromatic petroleum derived hydrocarbon resins and the hydrogenated derivatives thereof;
Aromatic modified cycloaliphatic resins and the hydrogenated derivatives thereof;
Polyterpene resins having a softening point from 95° C. to 140° C. the latter polyterpene resins generally resulting from the polymerization of terpene hydrocarbons, such as the mono-terpene known as pinene, in the presence of Friedel-Crafts catalysts at moderately low temperatures; the hydrogenated polyterpene resins are also included;
Copolymers and terpolymers of natural terpenes, e.g. styrene/terpene, α-methyl styrene/terpene and vinyl toluene/terpene; Natural and modified rosin such as, for example, gun rosin, wood rosin, tall-oil rosin, distilled rosin, hydrogenated rosin, dimerized rosin and polymerized rosin;
Glycerol and pentaerythritol esters of natural and modified rosin, such as, for example, the glycerol ester of pale wood rosin, the glycerol ester of hydrogenated rosin, the glycerol ester of polymerized rosin, the pentaerythritol ester of pale wood rosin, the pentaerythritol ester of hydrogenated rosin, the pentaerythritol ester of tall-oil rosin, and the phenolic modified pentaerythritol ester of rosin; and,
Phenolic-modified terpene resins such as for example, the resin product resulting from the condensation in an acidic medium of a terpene and a phenol.

In preferred embodiments, the resin is not modified by an unsaturated polycarboxylic acid, anhydride or ester (in other words, the composition does not comprise any resin modified by an unsaturated polycarboxylic acid, anhydride or ester).

Mixtures of two or more of the above described tackifying resins may also be used.

Tackifying resins which are useful for the present invention can include polar tackifying resins. However, the choice of available polar tackifying resins is limited in view of the fact that many of the polar resins appear only partially compatible with polyolefin polymers.

Alternatively, the tackifying resins can be selected from any of the nonpolar types, which are commercially available. One class of preferred resins are aliphatic petroleum hydrocarbon resins, examples of which are based on C5 olefins. Most preferred are nonpolar products which are hydrogenated di-cyclo-pentadiene (DCPD) based or aromatically modified derivatives thereof with softening points above 95° C. Examples of such resins are Escorez 5340, Escorez 5400 and Escorez 5600 sold by ExxonMobil Chemical company, Sukorez SU-120 sold by Kolon and JH6130 sold from Jinhai.

Preferably, the tackifying resin should have a Ring and Ball softening point (measured by ASTM E28) of at least 90° C. and preferably from 90° C. to 140° C. In general, the tackifying resin should be substantially aliphatic to insure compatibility between the resin and the polymer components.

Plasticizer

The hot melt adhesive composition of the present invention comprises a plasticizer. The plasticizer may be a solid or a liquid plasticizer.

The hot melt adhesive composition may comprise from 5 to 45%, preferably from 10 to 40%, more preferably from 25 to 35%, by weight of a plasticizer relative to the total weight of the composition.

The purpose of the plasticizer is to provide a desired viscosity control and to impart flexibility. A suitable plasticizer may be selected from the group which includes the usual plasticizing oils, such as mineral oils, but also olefin oligomers and low molecular weight polymers, as well as vegetable and animal oils and derivatives of such oils. The petroleum derived oils which may be employed are relatively high boiling materials containing only a minor proportion of aromatic hydrocarbons. The oligomers may be polypropylenes, polybutenes, hydrogenated polyisoprenes, hydrogenated polybutadienes, or the like having an average molecular weight between 350 and 10,000. Suitable vegetable and animal oils include glycerol esters of the usual fatty acids and polymerization products thereof. The plasticizers that are useful in the present invention can be any number of different plasticizers but mineral oils and liquid polybutenes having an average molecular weight less than 5,000 are particularly advantageous.

Typically, plasticizers are used to lower the viscosity of the overall hot melt adhesive composition without substantially decreasing the adhesive strength and/or service temperature of the adhesive composition as well as to extend the open time and to improve flexibility of the composition.

Weight Ratio Between the Plasticizer and the Total Polymer Components

The composition of the present invention presents a weight ratio between the plasticizer and the total polymer components of 2.6:1 or more. This is an advantageous ratio as it makes it possible to provide articles having a stable peel strength in a variety of conditions (such as temperature and add-on).

In one embodiment, the composition of the present invention presents a weight ratio between the plasticizer and the total polymer components of 2.6:1 to 10:1, preferably of 2.6:1 to 5:1.

Other Additives

The hot melt adhesive composition of the present invention also preferably includes at least one stabilizer (antioxidant).

The hot melt adhesive composition may comprise from 0.1 to 5%, preferably from 0.5 to 3%, by weight of a stabilizer relative to the total weight of the composition.

Suitable stabilizers are incorporated to help protect the polymers noted above, and thereby the total adhesive system from the effects of thermal and oxidative degradation which normally occurs during the manufacture and application of the adhesive, as well as in the ordinary exposure of the final product to the ambient environment.

Among the applicable stabilizers are high molecular weight hindered phenols and multifunction phenols, such as sulfur and phosphorous-containing phenols. Hindered phenols are well known to those skilled in the art and may be characterized as phenolic compounds that also contain sterically bulky radicals in close proximity to the phenolic hydroxyl group thereof. In particular, tertiary butyl groups generally are substituted onto the benzene ring in at least one of the ortho positions relative to the phenolic hydroxyl group. The presence of these sterically bulky substituted radicals in the vicinity of the hydroxyl group serves to retard its stretching frequency and correspondingly, its reactivity; this steric hindrance thus providing the phenolic compound with its stabilizing properties. Representative hindered phenols include: 1,3,5-trimethyl-2,4,6-tris(3-5-di-tert-butyl-4-hydroxybenzyl) benzene; pentaerythritol tetrakis-3(3,5-di-tert-butyl-4-hydroxyphenyl) propionate; n-octadecyl-3(3,5-di-tert-butyl-4-hydroxyphenyl) propionate, 4.4′-methylenebis(4-methyl-6-tert-butylphenol); 2,6-di-tert-butylphenol; 6-(4-hydroxyphenoxy)-2,4-bis(n-octylthio)-1,3,5-triazine; 2,3,6-tris(4-hydroxy-3,5-di-tert-butyl-phenoxy)-1,3,5-triazine; di-n-octadecyl-3,5-di-tert-butyl-4-hydroxybenzylphosphonate; 2-(n-octyl-thio)ethyl-3,5-di-tert-butyl-4-hydroxybenzoate and sorbitol hexa-3(3,5-di-tert-butyl-4-hydroxy-phenyl)propionate.

Especially preferred as a stabilizer is pentaerythritol tetrakis-3(3,5-di-tert-butyl-4-hydroxyphenol) propionate.

The performance of these stabilizers may be further enhanced by utilizing, in conjunction therewith; (1) synergists such as, for example, thiodipropionate esters and phosphites; examples of these include dialuryl thiodipropionate (DLTDP) and tris(nonylphenyl)phosphite (TNPP), respectively; and (2) chelating agents and metal deactivators as, for example, ethylenediaminetetraacitic acid, salts thereof and disalicylalpropylenediimine.

The hot melt adhesive composition according to the present invention may further include at least one wax. The hot melt adhesive composition may comprise from 0 to 30%, preferably from 0.5 to 3%, by weight of wax relative to the total weight of the composition.

The presence of the wax makes it possible to reduce the melt viscosity of the hot melt adhesive without appreciably decreasing its adhesive bonding characteristics. These waxes are also used to reduce the open time, or set-up time of the composition without affecting the temperature performance.

Among useful wax materials are:
low molecular weight, that is, number average molecular weight (Mn) from 100 to 6000 g/mol, polyethylene wax having a hardness value, as determined by ASTM method D-1321, from 0.1 to 120 and ASTM softening points from 65° C. to 140° C.;
petroleum waxes such as paraffin wax having a melting point from 50° C. to 80° C. and microcrystalline wax having a melting point from 55° C. to 100° C., the latter melting points being determined by ASTM method D127-60; metallocene catalyzed propylene-based wax like those commercialized by Clariant under the name “Licocene”; metallocene catalyzed wax or single-site catalyzed wax like for example those described in U.S. Pat. Nos. 4,914,253, 6,319,979, WO 97/33921 or WO 98/03603;
synthetic waxes made by polymerizing carbon monoxide and hydrogen such as Fischer-Tropsch wax; and polyolefin waxes. As used herein, the term “polyolefin wax” refers to those polymeric or long-chain entities comprised of olefinic monomer units. These materials are commercially available from Westlake Chemical Co. under the trade name “Epolene”.

Preferably, the wax materials used have a Ring and Ball softening point from 93° C. to 177° C. As should be understood, each of these waxes is solid at room temperature. Other useful substances include hydrogenated animal, fish and vegetable fats and oils such as hydrogenated tallow, lard, soy oil, cottonseed oil, castor oil, menhadin oil, cod liver oil, and the like, and which are solid at ambient temperature by virtue of their being hydrogenated, have also been found to be useful with respect to functioning as a wax material equivalent. These hydrogenated materials are often referred to in the adhesives industry as “animal or vegetable waxes”. According to some embodiments, the composition of the present invention may comprise an acid-modified component. Such compounds may include for example a wax, a resin (for example a petroleum or a tackifier resin), a softening agent, an oil, a plasticizer or a rubber, preferably a resin. In other words, the composition of the present invention may comprise a component (preferably a resin) modified by an unsaturated polycarboxylic acid, anhydride or ester (therefore comprising one or more acidic groups). Examples of polycarboxylic acids include maleic acid, fumaric acid, itaconic acid, citraconic acid and polycarboxylic acid. An example of such compound may be the acid-modified resin Quintone D295 sold by Zeon. According to some embodiments, other optional auxiliary additives may be incorporated in the hot melt adhesive composition, in order to modify one or more physical properties of the composition. The hot melt adhesive composition may comprise from 0.01 to 3% by weight of auxiliary additives relative to the weight of the composition.

Such additives may include, for example, inert colorants such as titanium dioxide as well as fillers, surfactants, other types of polymers, crosslinking agents, nucleating agents, reactive compounds, fire-retardant mineral or organic agents, ultraviolet (UV) or infrared (IR) light absorbing agents, and UV or IR fluorescing agents. Typical fillers include talc, calcium carbonate, clay, silica, mica, wollastonite, feldspar, aluminum silicate, alumina, hydrated alumina, glass microspheres, ceramic microspheres, thermoplastic microspheres, baryte and wood flour. These optional auxiliary additives are well known in this art.

In a preferred embodiment, the composition of the present invention does not comprise butyl rubber.

In a preferred embodiment, the composition of the present invention comprises:
from 5 to 20%, preferably from 6 to 17%, more preferably from 7 to 14%, by weight of the first polymer component relative to the total weight of the composition;
a second polymer as defined above;
from 35 to 75%, preferably from 45 to 70%, more preferably from 50 to 60%, by weight of a tackifying resin relative to the total weight of the composition;
from 5 to 45%, preferably from 10 to 40%, more preferably from 25 to 35%, by weight of a plasticizer relative to the total weight of the composition;

wherein the composition comprises a weight ratio between the first polymer component and the second polymer component from 3:2 to 10:1, more preferably from 2:1 to 8:1; and wherein the composition comprises a weight ratio between the plasticizer and the total polymer components of 2.6:1 or more, preferably of 2.6:1 to 10:1, even more preferably of 2.6:1 to 5:1.

Viscosity

The hot melt adhesive composition of the present invention may have a viscosity from 1,000 to 40,000 mPa·s, preferably from 5,000 to 35000 mPa·s at a temperature of 120° C. The viscosity can be measured according to the ASTM D3236 standard in a Brookfield RVT viscometer (spindle #27).

Softening Point

The hot melt adhesive composition of the present invention may have a softening point from 70 to 120° C., preferably from 85 to 110° C. The softening point can be measured according to the ASTM E-28 standard.

Peel Strength

The hot melt adhesive composition of the present invention makes it possible to obtain a stable peel in a variety of conditions. More particularly, the hot melt adhesive composition makes it possible to obtain articles having a peel strength that remains relatively stable over increasing temperature and increasing add-on. By “add-on” is meant the weight of the coating formed by the hot melt adhesive composition. By “relatively stable” is meant that the coefficient in variation of the peel strength depending on the temperature is lower than 0.4 and the coefficient in variation of the peel strength depending on the add-on is also lower than 0.4.

Thus, the coefficient in variation of the peel strength depending on the temperature may be lower than 0.4. For example, the coefficient in variation of the peel strength depending on the temperature may be from 0.1 to 0.15; or from 0.15 to 0.2; or from 0.2 to 0.25; or from 0.25 to 0.3; or from 0.3 to 0.35; or from 0.35 to less than 0.4.

In addition, the coefficient in variation of the peel strength depending on the add-on may also be lower than 0.4. For example, the coefficient in variation of the peel strength depending on the add-on may be from 0.1 to 0.15; or from 0.15 to 0.2; or from 0.2 to 0.25; or from 0.25 to 0.3; or from 0.3 to 0.35; or from 0.35 to less than 0.4.

Temperature-dependency and add-on-dependency coefficient in variation is measured as indicated below:

The hot melt adhesive composition is first heated at a temperature from 140 to 150° C. and is then applied to a primary substrate which is a release paper at 20, 30, 40 and 50 g/m² coating weight by slot coating application with 0.3 seconds open time and 0.01 MPa compression at the nip rolls. The pattern of the hot-melt adhesive composition has 13 stripes (lines) each stripe being 2 mm wide. The interval between the stripes are 2 mm. A secondary substrate (non-breathable polyethylene (PE) film) is then laminated with the primary substrate comprising the composition. The composition is transferred to the non-breathable PE film side to prepare a sample. The laminate is then stored at 23° C. under an atmosphere of 50% relative humidity for 24 hours to cool and solidify the hot melt adhesive. The laminate is then cut in order to obtain test specimens having a width of 50 mm and a length of 100 mm in a cross direction.

For measuring peel strength at 40° C.: The test specimens and cotton are stored under an environment of 40° C. for 3 hours. After removing the release paper from the test specimen, the test specimen is applied to the cotton and pressurized by a 2 kg roller back and forth. After that, the T-peel test is performed using a Tensilon tester at a rate of 300 mm/min at 40° C. and the initial peel strength is measured.

For measuring peel strength at 0, 23 and 30° C., the same procedure can be followed at 0, 23 and 30° C. respectively.

For measuring temperature-dependency coefficient in variation: An average peel strength is calculated for each coating weight (20, 30, 40 and 50 g/m²).

A standard deviation was also calculated for each coating weight (20, 30, and 50 g/m²).

The coefficient in variation is calculated by dividing the standard deviation of each coating weight with the average peel strength of each and respective coating weight.

For measuring add-on-dependency coefficient in variation: An average peel strength is calculated for each temperature (0, 23, 30 and 40° C.). A standard deviation was also calculated for each temperature (0, 23, 30 and 40° C.).

The coefficient in variation is calculated by dividing the standard deviation of each temperature with the average peel strength of each and respective temperature.

Hot Melt Adhesive Composition (Product by Process)

The hot melt adhesive composition may be obtained by mixing a first polymer component, a second polymer component, a tackifying resin, a plasticizer and any additional components, as described hereinbefore.

Use

In a second aspect, the present invention relates to the use of the hot melt adhesive compositions as described above, for coating a substrate or for bonding two substrates together.

Each substrate to be coated or bonded may comprise a film, nonwoven material or woven material. It may comprise absorbent fluff, super absorbent polymer (SAP), composite material, plastics which may be elastomeric or non-elastomeric, for example styrene block copolymers (SBC), polyurethane, and polyolefin, and any mixture thereof. Preferably, the substrate is a nonwoven fabric. In this case, the process of manufacturing an assembly product (or laminate) may comprise:
a step (i) of heating the hot melt adhesive composition according to the invention, e.g. at a temperature ranging from 120° C. to 180° C., for at least a period of time long enough to render the hot melt adhesive composition liquid enough to be applied on a substrate (for example at least two hours), then
a step (ii) of coating said composition on a first substrate, then
a step (iii) of putting into contact the coated surface of the first substrate with the surface of a second substrate, so as to form an adhesive joint bonding the two substrates.

The substrates may be different or of the same nature, with various forms (layer or film, strands, fluff).

Article

In a third aspect, the invention relates to an article comprising at least one interior or exterior surface coated with the hot melt adhesive composition as described above. In particular embodiments, the present invention relates to an assembly product comprising at least two substrates bonded by at least one hot melt adhesive composition according to the invention.

The coated surface and the bonded substrates may be as described above.

The hot melt adhesive composition according to the invention may be used as the laminating adhesive to bind a plurality of substrate layers for example to manufacture toilet tissues, paper towels, wipes and other consumer products, particularly absorbent articles such as disposable hygiene products, and more particularly disposable diapers.

In some embodiments, the assembly product according to the invention may be a multilayer product comprising at least two layers of substrate(s) bonded by at least one hot melt adhesive composition according to the invention.

In the assembly product according to the invention, the at least two layers of substrate(s) may be joined adhesively by a layer of the hot melt adhesive composition according to the invention, sandwiched between the two layers of substrate(s).

Alternatively or cumulatively, the at least two layers of substrate(s) may be adhesively joined by spots of the hot melt adhesive composition according to the invention.

Preferably, the article/assembly product is a disposable nonwoven absorbent article. Mention can be made of disposable diapers, disposable training pants, disposable adult incontinent pads or briefs and disposable feminine sanitary napkins or pads, surgical masks and surgical coats.

### EXAMPLES

The following examples illustrate the invention without limiting it.

Eight compositions were prepared in order to study the temperature-dependency and add-on-dependency coefficient in variation. Compositions A to D are according to the invention while compositions E to H are comparative compositions.

The compositions A to H comprise some of the following compounds:
SEBS1: Taipol® SEBS 6153 supplied by TSRC Corporation/Dexco Polymers comprising a styrene content of 29%, a diblock content of 0% and a viscosity of a 10% by weight toluene solution at 30° C. of 55 mPa·s (corresponding to the first polymer component).
SEBS2: Kraton® G-1726 available from Kraton comprising a styrene content of 30%, a diblock content of 70% and a viscosity of a 10% by weight toluene solution at 30° C. of 10 mPa·s (corresponding to the second polymer component).
SEBS3: Kraton® G-1657 available from Kraton comprising a styrene content of 13%, a diblock content of 29% and a viscosity of a 10% by weight toluene solution at 30° C. of 65 mPa·s (corresponding to the second polymer component).
R1: Sukorez® SU-120 available from Kolon comprising a fully hydrogenated DCPD resin.
R2: JH6130 available from Jinhai comprising a hydrogenated DCPD resin.
R3: Quintone® D295 available from Zeon comprising an acid-modified non-hydrogenated petroleum resin. This resin is modified with maleic acid and has a softening point of 94° C. and an acid value of 8.0 mg KOH/g.
P1: Apar Poweroil® H350 available from Apar comprising a paraffinic oil plasticizer.
A1: Antioxidant Irganox® 1010 being a hindered phenol type antioxidant available from BASF.

| Compositions | A | B | C | D | E | F | G | H |
|---|---|---|---|---|---|---|---|---|
| SEBS1 (%) | 9.5 | 9.5 | 9.5 | 9.5 | 13 | 13 | 11.5 | 10 |
| SEBS2 (%) | — | — | — | — | — | 3 | 3 | — |
| SEBS3 (%) | 3 | 3 | 3 | 3 | — | — | — | 3 |
| R1 (%) | — | — | 54.5 | — | 57.2 | 57.2 | — | 57 |
| R2 (%) | 54.5 | 51.5 | — | 52.5 | — | — | 52.5 | |
| R3 (%) | — | 3 | — | — | — | — | — | — |
| P1 (%) | 32.5 | 32.5 | 32.5 | 34.5 | 29.5 | 26.5 | 32.5 | 29.5 |
| A1 (%) | 0.5 | 0.5 | 0.5 | 0.5 | 0.5 | 0.5 | 0.5 | 0.5 |
| Plasticizer/ | 2.6 | 2.6 | 2.6 | 2.76 | 2.27 | 1.66 | 2.24 | 2.27 |
| polymers | | | | | | | | |

The compositions A to H were prepared by placing the components listed in the table above into a jacketed mixing kettle equipped with a heater and a stirrer, and then mixed while heating at 170° C. for 120 min under vacuum to remove any entrapped air.

The viscosity of the compositions A to H was measured according to the ASTM D3236 standard in a Brookfield RVT viscometer.

The softening point of the compositions A to H was measured according to the ASTM E-28 standard.

The results are disclosed in the table below:

| | Viscosity (mPa · s) at | |
|---|---|---|
| Compositions | 120° C. | Softening point (° C.) |
| A | 18060 | 96.6 |
| B | 15810 | 96.3 |
| C | 10150 | 91.7 |
| D | 15500 | 95.9 |
| E | 17690 | 96.8 |
| F | 32000 | 97.8 |
| G | 26380 | 100.3 |
| H | 13750 | 93.8 |

The temperature-dependency and add-on-dependency coefficient in variation (CV) were measured as detailed in the above description.

| Compositions | A | B | C | D | E | F | G | H |
|---|---|---|---|---|---|---|---|---|
| Temperature- | 0.29 | 0.33 | 0.25 | 0.34 | 0.39 | 0.65 | 0.35 | 0.43 |
| dependency | | | | | | | | |
| CV at 20 g/m² | | | | | | | | |
| Temperature- | 0.36 | 0.33 | 0.30 | 0.36 | 0.41 | 0.52 | 0.34 | 0.38 |
| dependency | | | | | | | | |
| CV at 30 g/m² | | | | | | | | |
| Temperature- | 0.34 | 0.32 | 0.25 | 0.30 | 0.44 | 0.55 | 0.27 | 0.41 |
| dependency | | | | | | | | |
| CV at 40 g/m² | | | | | | | | |
| Temperature- | 0.38 | 0.36 | 0.23 | 0.34 | 0.47 | 0.56 | 0.31 | 0.43 |
| dependency | | | | | | | | |
| CV at 50 g/m² | | | | | | | | |
| Add-on- | 0.22 | 0.17 | 0.22 | 0.22 | 0.15 | 0.29 | 0.28 | 0.26 |
| dependency | | | | | | | | |
| CV at 0° C. | | | | | | | | |
| Add-on- | 0.28 | 0.20 | 0.26 | 0.23 | 0.29 | 0.19 | 0.32 | 0.28 |
| dependency | | | | | | | | |
| CV at 23° C. | | | | | | | | |
| Add-on- | 0.37 | 0.25 | 0.31 | 0.25 | 0.27 | 0.25 | 0.38 | 0.28 |
| dependency | | | | | | | | |
| CV at 30° C. | | | | | | | | |
| Add-on- | 0.32 | 0.24 | 0.29 | 0.28 | 0.34 | 0.24 | 0.45 | 0.28 |
| dependency | | | | | | | | |
| CV at 40° C. | | | | | | | | |

As can be seen from the table above, contrary to the comparative compositions (E to H), the compositions according to the present invention (A to D) make it possible to achieve at the same time good temperature dependency at all coating weight (CV lower than 0.4) and a good add-on dependency CV at all temperatures (CV lower than 0.4).

1-18. (canceled)

19. A hot melt adhesive composition comprising:
a first polymer component comprising at least one hydrogenated linear styrene triblock copolymer;
a second polymer component comprising at least one hydrogenated linear styrene triblock copolymer having a diblock content from 10 to 90%;
at least one tackifying resin; and
at least one plasticizer;
wherein the composition comprises a weight ratio between the first polymer component and the second polymer component of 1:1; and
wherein the composition comprises a weight ratio between the plasticizer and the total polymer components of 2.6:1 or more.

20. The composition according to claim 19, wherein the hydrogenated linear styrene triblock copolymer of the first polymer component is selected from the group consisting of SEBS, SEPS, SEEPS, SBBS and blends thereof.

21. The composition according to claim 19, wherein the hydrogenated linear styrene triblock copolymer of the second polymer component is selected from the group consisting of SEBS, SEPS, SEEPS, SBBS, and blends thereof.

22. The composition according to claim 19, wherein the polymer components have a viscosity of a 10% by weight toluene solution at 30° C. of 100 mPa·s or less.

23. The composition according to claim 19, wherein the first polymer component has a styrene content from 10 to 50%.

24. The composition according to claim 19, wherein the second polymer component has a styrene content from 5 to 50%.

25. The composition according to claim 19, wherein it comprises at least one additional polymer component comprising an additional styrene block copolymer selected from the group consisting of SB, SI, SBS, SIS, and SIBS.

26. The composition according to claim 19, wherein it comprises a coefficient in variation of the peel strength depending on the temperature being lower than 0.4.

27. The composition according to claim 19, wherein it comprises a coefficient in variation of the peel strength depending on the add-on being lower than 0.4.

28. The composition according to claim 19, wherein it is obtained by mixing the first polymer component, the second polymer component, the tackifying resin and the plasticizer.

29. The composition according to claim 19, wherein it does not comprise butyl rubber.

30. The composition according to claim 19, wherein it comprises from 5 to 20%, by weight of the first polymer component relative to the total weight of the composition.

31. The composition according to claim 19, wherein it comprises from 35 to 75%, by weight of a tackifying resin relative to the total weight of the composition.

32. The composition according to claim 19, wherein the weight ratio between the plasticizer and the total polymer components is of 2.6:1 to 10:1.

33. An adhesive comprising the composition according to claim 19, wherein the adhesive is used for bonding two substrates together, or as a coating on the surface of a substrate.

34. An article comprising at least one interior or exterior surface coated with the hot melt adhesive composition according to claim 19.

35. The article of claim 34, wherein the interior or exterior surface is a nonwoven fabric.

36. The article according to claim 34, being selected from the group consisting of a disposable diaper, disposable training pants, a feminine sanitary napkin, an adsorbent pad, a surgical mask, and a surgical coat.
